(19) United States
(12) Patent Application Publication
(10) Pub. No.: US 2019/0129508 A1
(43) Pub. Date: May 2, 2019

(54) Method and System for Interacting with a Wearable Electronic Device

(71) Applicant: CARNEGIE MELLON UNIVERSITY, Pittsburgh, PA (US)

(72) Inventors: Christopher Harrison, Pittsburgh, PA (US); Robert Xiao, Pittsburgh, PA (US); Gierad Laput, Pittsburgh, PA (US)

(73) Assignee: CARNEGIE MELLON UNIVERSITY, Pittsburgh, PA (US)

(21) Appl. No.: 16/094,502

(22) PCT Filed: Jun. 23, 2017
(86) PCT No.: PCT/US17/39131

§ 371 Date: Oct. 18, 2018

Related U.S. Application Data
(60) Provisional application No. 62/493,163, filed on Jun. 23, 2016.

(51) Int. Cl. G06F 3/01; A61B 5/00; A61B 5/11; G06F 3/0346; G04G 21/02

(52) U.S. Cl. CPC G06F 3/017; A61B 5/0028; A61B 5/1123; A61B 5/681; A61B 2562/0219; G06F 3/0346; G04G 21/025; A61B 2503/12; G06F 3/015

(57) ABSTRACT

Disclosed herein is a method of interacting with a wearable electronic device. The wearable electronic device, comprising a vibration sensor, captures vibrations transmitted through a body part on which the electronic device is worn. The vibration can emanate from an object in contact with the user's body or by the motions of the body itself. Once received by the wearable electronic device, the vibrations are analyzed and identified as a specific object, data message, or movement.

### CROSS-REFERENCE TO RELATED APPLICATIONS

This application claims the benefit under 35 U.S.C. § 119 of Provisional Application Ser. No. 62/493,163, filed Jun. 23, 2016, which is incorporated herein by reference.

### STATEMENT REGARDING FEDERALLY SPONSORED RESEARCH

Not Applicable.

### BACKGROUND OF THE INVENTION

The invention relates to a method of interacting with a wearable electronic device. Wearable electronic devices are unique among computing devices in that they are worn, offering great potential to transform arms, hands, and other body parts into expressive input and sensing platforms. For example, with smartwatches, tiny micro-vibrations propagate through the arm as people use their hands, carrying information about the objects they interact with and the activities they perform throughout the day. Smartwatches and other wearables are ideally situated to capture these vibrations.

Although most modern wearable electronic devices contain accelerometers and other sensors capable of capturing vibrations, they are generally limited to sensing coarse movements with a sampling rate of around 100 Hz. This is sufficient for their main use, which is detecting the orientation of the device (e.g., to automatically activate the screen when raised), but is generally not robust enough to allow user interaction through hand gestures or object detection, for example. In addition to accelerometers, most devices include microphones, which provide even higher sampling rates (typically 44.1 kHz). However, microphones are specifically designed to capture airborne vibrations, not contact vibrations, which means purposeful signals must be segmented from background environmental noise.

Prior attempts have been made for sensing hand gestures. For example, a popular approach for hand gesture recognition takes advantage of optical sensors such as cameras and IR sensors. It is also possible to sense hand gestures by approximating skin contours and deformations. For instance, armbands instrumented with IR sensors or pressure sensors can measure skin contact variations whenever particular gestures are performed. Despite being low-cost, these approaches are highly dependent on contact conditions, which are inherently sensitive to periodic armband removal, and equally susceptible to unintentional arm movements.

Hand gestures can likewise be modeled by examining the internal anatomical configuration of the user's arm. Approaches can be passive, such as electromyography, where gestures are classified by measuring the electrical signals caused by muscle activation, or active, where a signal is injected into the body to detect hand gestures.

Finally, coarse and fine hand gestures indirectly induce arm motions which can be captured by inertial sensors e.g., accelerometers and gyroscopes. Previous work introduced gloves equipped with accelerometers to model fine hand gestures. Likewise, several techniques take advantage of the inertial sensors present in contemporary smartwatches. However, the approaches utilize wearable accelerometers to recognize gross-motor or whole hand motions. In alternative approaches, finger gesture recognition was accomplished using commodity accelerometers on a smartwatch, but this approach utilized low frequency vibrations and the technique is highly sensitive to arm orientation, and was never deployed in a real-time environment.

Bio-acoustics has been studied in many fields, including human-computer interaction (HCI). For instance, in one method, contact microphones are placed on the user's wrist to capture gross finger movement. In another method, the user's limbs are instrumented with piezo sensors to detect gestures (e.g., finger flick, left foot rotate). Another method leveraged a similar technique, using an array of piezo sensors strapped onto the user's arm (above and below the elbow). These bio-acoustic sensing approaches rely heavily on special-purpose sensors, increasing their invasiveness and ultimately limiting their practicality.

Object recognition offers relevant information more closely matching a user's immediate context and environment. However, most approaches rely on markers or special-purpose tags. These offer robust recognition, but ultimately require every object to be instrumented. Further, these approaches approximate whether an object is nearby, and not when it is truly grasped or handled. Prior work has also leveraged acoustics to recognize objects. For example, in one method, a worn necklace equipped with an accelerometer and a microphone was used to classify workshop tools, although the approach was susceptible to background noise.

Wearable devices are also increasingly being used for object sensing and recognition. One technique utilized magnetic sensors and hand-worn coils to identify objects based on magnetic field changes. Another technique offered a similar approach, using three magneto-inductive sensors to identify objects during regular operation. Magnetic induction relies heavily on proximate contact between the sensor and the object, which is affected by posture, hand orientation, or even the inherent magnetic noise present in the human body. It is also possible to characteristically identify objects solely based on unintentionally emitted electromagnetic (EM) noise.

Data transmission through the body has been successfully demonstrated with radio frequency (RF) waves, in the form of “personal area networks.” Such networks can successfully transmit data at very high speeds amongst specially-equipped devices near the body. Other systems use vibroacoustics to transmit data. For example, one system using an accelerometer and vibration motor mounted to a cantilevered metal arm (to amplify vibrations), transmitted data at about 200 bits/sec. By way of further example, AT&T Labs publicly demonstrated a system that transmitted bio-acoustic data using a piezoelectric buzzer, although the technical details have not been published. These systems do not quite reach the level of unobtrusive, wearable electronics.

As discussed, many methods and systems have been developed to allow interaction with wearable devices or to allow users to interact with their environment. However, prior approaches require specialized equipment or provide only limited interactivity. It would therefore be advantageous to develop a method of interacting with a wearable electronic device that overcomes the drawbacks of the prior art.

### BRIEF SUMMARY

According to embodiments of the present invention is a method and system for interacting with a wearable electronic device. Wearable electronic devices, such as smartwatches, are unique in that they reside on the body, presenting great potential for always-available input and interaction. Smartwatches, for example, are ideal for capturing bio-acoustic signals due to their location on the wrist. In one embodiment of the present invention, the sampling rate of a smartwatch's existing accelerometer is set to about 4 kHz, capturing high-fidelity data on movements of the hand and wrist. This high sampling rate allows the wearable to not only capture coarse motions, but also rich bio-acoustic signals. With this bio-acoustic data, the wearable electronic device can be used to classify hand gestures such as flicks, claps, scratches, and taps, which combine with on-device motion tracking to create a wide range of expressive input modalities. Bio-acoustic sensing can also detect the vibrations of grasped mechanical or motor-powered objects, enabling passive object recognition that can augment everyday experiences with context-aware functionality. In alternative embodiments, structured vibrations from a transducer can be transmitted through the body to the wearable, increasing the interactive possibilities.

As will be discussed, the method of the present invention can be applied to a wide array of use domains. First, bio-acoustic data can be used to classify hand gestures, which are combined with on-device motion tracking to enable a wide range of expressive input modalities. Second, vibrations of grasped mechanical or motor-powered objects are detected and classified, enabling un-instrumented object recognition. Finally, structured vibrations are used for reliable data transmission through the human body. The method and system of the present invention are accurate, robust to noise, relatively consistent across users, and independent of location or environment.

### BRIEF DESCRIPTION OF THE SEVERAL VIEWS OF THE DRAWINGS

FIG. 1 is a block diagram showing the system according to one embodiment.

FIG. 2 is a block diagram showing the system according to an alternative embodiment.

FIGS. 3A-3D show captured accelerometer signals at different sampling rates.

FIGS. 4A-4B show interaction with a watch and a graph depicting a resonance profile.

FIG. 5 is a chart showing various hand gestures and their accompanying vibration profile.

FIG. 6 is a flow diagram depicting the method of the present invention, according to one embodiment.

FIG. 7 is a diagram showing various gestures and interaction modalities.

FIG. 8 depicts various objects and their corresponding bio-acoustic signal.

FIGS. 9A-9B show a data transmission received by a wearable electronic device, according to a method of one embodiment of the present invention.

FIG. 10 is a chart of different modulation schemes.

FIGS. 11A-11H depict various interactions with a wearable device.

### DETAILED DESCRIPTION

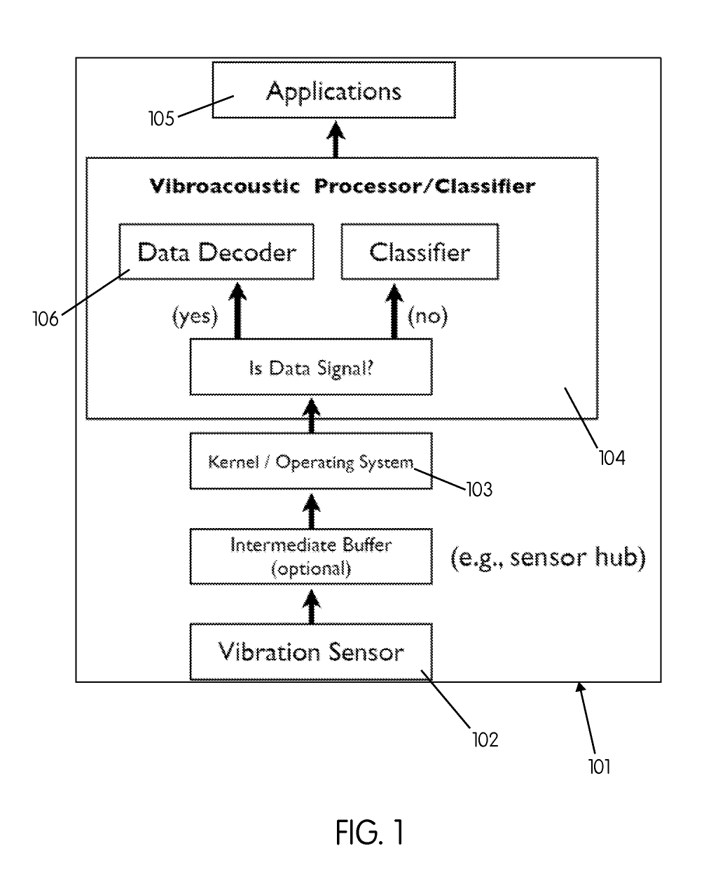

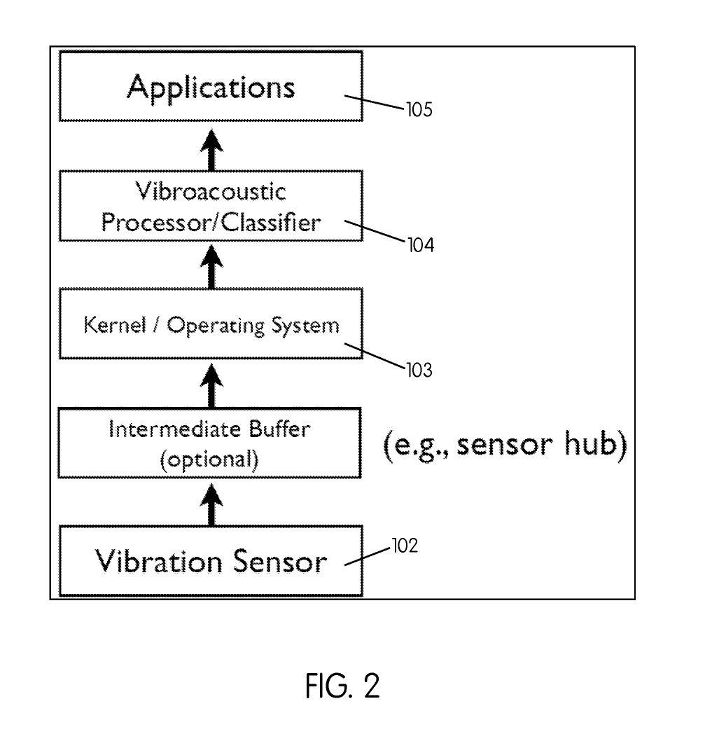

According to embodiments of the present invention is a method and system for interacting with a wearable electronic device 101. As shown in FIG. 1, the wearable 101 comprises an inertial measurement unit (IMU) or vibration sensor 102, such as an accelerometer or gyroscope, and software, such as a kernel/operating system 103, classifier 104, applications 105, and a data decoder 106. Additional sensors may also be present. In the embodiments shown in FIGS. 1-2, the components of the wearable device (except the vibration sensor 102) may comprise software, firmware, dedicated circuitry, or any combination of hardware and software.

The applications 105 include user interfaces that can be launched once a gesture or object is recognized. For example, if a user grasps an electronic toothbrush, the wearable 101 will launch a timer to ensure the user brushes for an appropriate amount of time. FIG. 2 shows and alternative embodiment of the wearable electronic device 101, in which a data decoder 106 is not present. This embodiment can be used when the user does not expect to utilize data transmission.

Although most wearable electronic devices 101 (including smartwatches, activity trackers, and other devices designed to be worn on the body) contain capable IMU's 102, existing software for these devices 101 generally limit accelerometer data access to about 100 Hz. This rate is sufficient for detecting coarse movements such as changes in screen orientation or gross interactions such as walking, sitting, or standing. However, these IMU's 102 often support significantly higher sample rates—up to thousands of hertz. At these faster sampling speeds, the wearable 101 can capture nuanced and fine-grained movements that are initiated or experienced by the human user. Like water, the human body is a non-compressible medium, making it an excellent vibration carrier. For example, when sampling at 4000 Hz, vibrations oscillating up to 2000 Hz (e.g., gestures, grasped objects) can be sensed and identified (per the Nyquist Theorem). This superior sensitivity transforms the wearable 101 into a bioacoustic sensor capable of detecting minute compressive waves propagating through the human body.

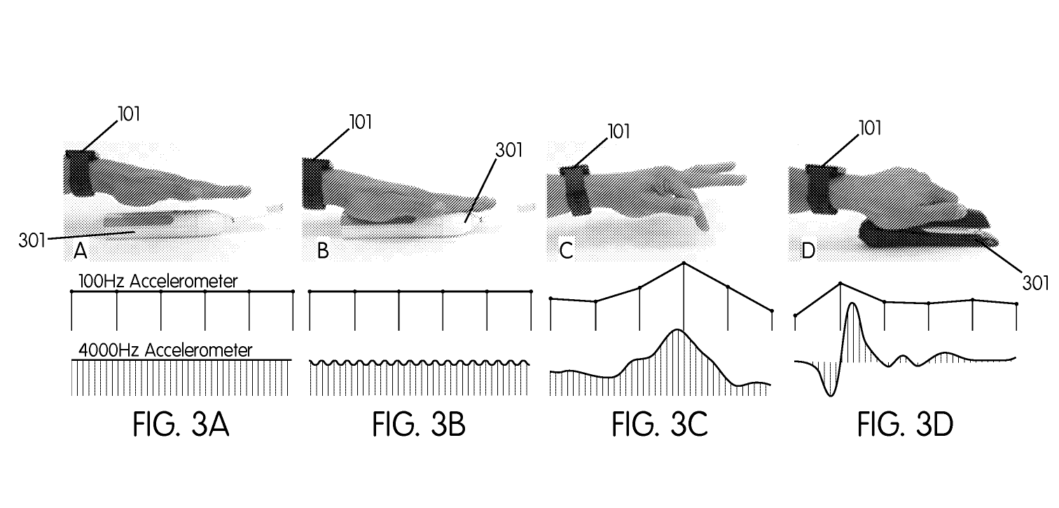

For example, FIGS. 3A-3D show a comparison of 100 Hz vs. 4000 Hz accelerometer signals. At steady state, both signals look identical, as shown in FIG. 3A. However, high frequency micro-vibrations propagating through the arm are missed by the 100 Hz accelerometer (FIG. 3B), whereas the sinusoidal oscillations of a toothbrush's motor are clearly visible at a 4000 Hz sampling rate. Characteristic vibrations can come from oscillating objects, hand gestures (FIG. 3C), and the operation of mechanical objects (FIG. 3D). The 100 Hz signal captures the coarse impulse, but no useful spectral information is available. Each activity and object produces characteristic vibroacoustic signatures, and more critically, were only captured when in contact with the hand or other body part of the user. These high-fidelity signals resemble those captured by a microphone, yet lack any audible external noise.

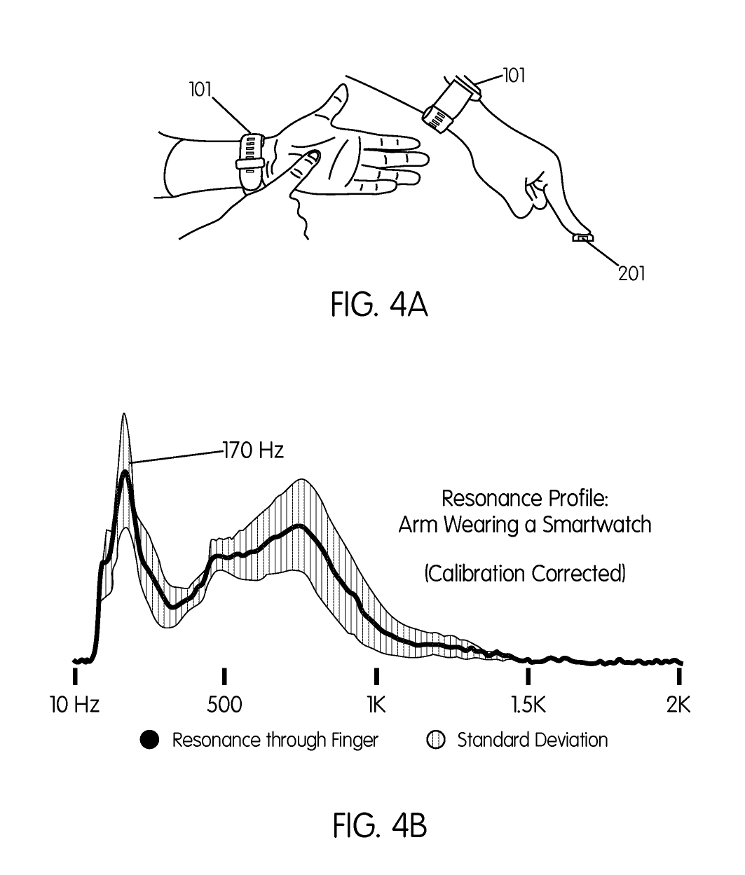

Like any medium, the human arm characteristically amplifies or attenuates vibrations at different frequencies. Therefore, certain frequencies transmit more easily through the human body. FIG. 4A depicts an example of a user with a watch 101 placed on their wrist, with FIG. 4B showing a resonance profile for this type of configuration (calibrated, watch+arm). Vibration frequencies between 20 Hz and 1 kHz transmit particularly well through the arm, with salient peaks at ˜170 Hz and ˜750 Hz. With this knowledge, the wearable 101 can be tuned for optimal performance.

In one example embodiment, the wearable electronic device 101 comprises an LG G W100 smartwatch. The smartwatch, in this example, includes an InvenSense MPU6515 IMU 102 capable of measuring acceleration at 4000 samples per second. This type of IMU 102 can be found in many popular smartwatches and activity trackers. Despite the high sampling rate capability, the maximum rate obtainable through the Android Wear API is 100 Hz. Therefore, to detect user movements, the Linux kernel 103 on the device must be modified, replacing the existing accelerometer driver with a custom driver.

In the example using a smartwatch, the kernel driver interfaces with the IMU 102 via an inter-integrated circuit (I²C), configuring the IMU 102 registers to enable its documented high-speed operation. Notably, this requires the system to use the IMU's 102 onboard 4096-byte FIFO to avoid excessively waking up the system CPU. However, this FIFO only stores 160 ms of data—each data sample consists of a 16-bit sample for each of the three axes. Thus, the driver is configured to poll the accelerometer in a dedicated kernel thread, which reads the accelerometer FIFO into a larger buffer every 50 ms. Overall, the thread uses about 9% of one of the wearable's 101 four CPU cores.

To improve the accuracy of systems with internal clocks that are not temperature-stabilized, a correction is made. For non-corrected clocks, higher sampling rates are experienced as the CPU temperature increased. For example, sampling rates may vary between 3990 Hz (watch sleeping, off wrist) to 4080 Hz (on arm, high CPU activity). To correct this error, in one embodiment the kernel driver is augmented to compute the rate at which samples were written into the MPU's FIFO buffer using a nanosecond-precision kernel timestamp. For applications requiring precise sampling rates, such as resonance profiling and data transmission, the input data is normalized to 4000 Hz using a sine-based interpolator capable of supporting continuously variable input sample rates.

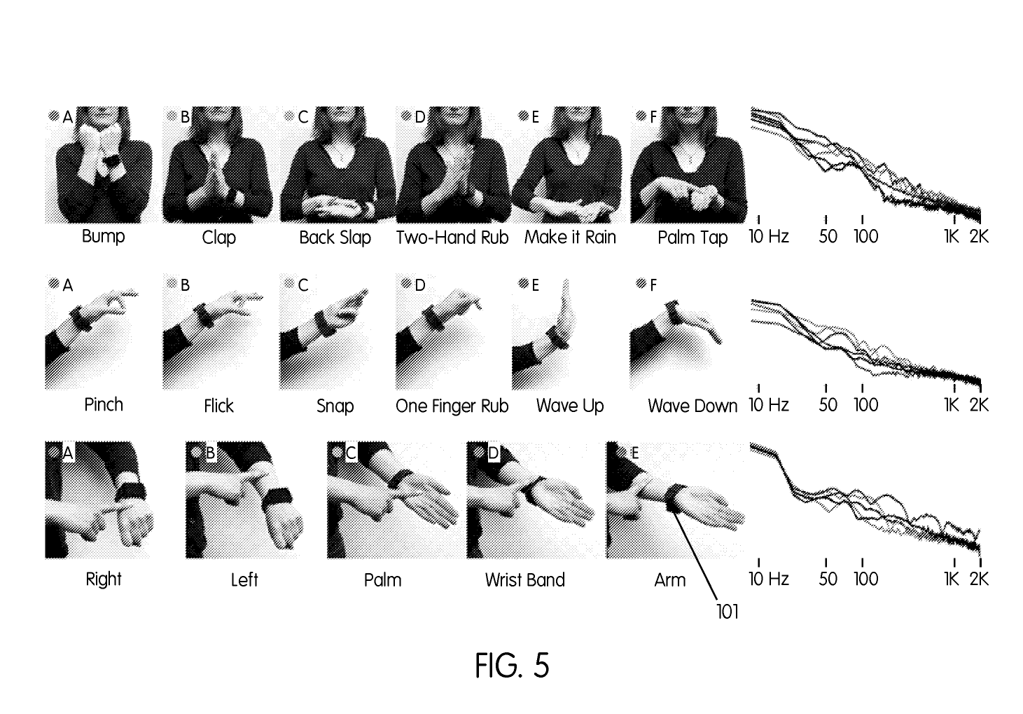

In one example method of interacting with the wearable electronic device 101, unique hand gestures, such as flicks, claps, snaps, scratches and taps performed by a user are detected and classified by the wearable 101. Each gesture is then classified by recognizing the distinctive micro-vibrations created by the movement and propagated through the arm. Depending on the location and type of gesture, different frequencies of vibrations are generated. Subsequently, various frequencies are attenuated during propagation (e.g., anatomical features can act as passive vibroacoustic filters). The resulting frequency profiles make many gestures uniquely identifiable. Many types of gestures can be recognized, such as one-handed gestures, two-handed gestures, and on-body touch input (see FIG. 5).

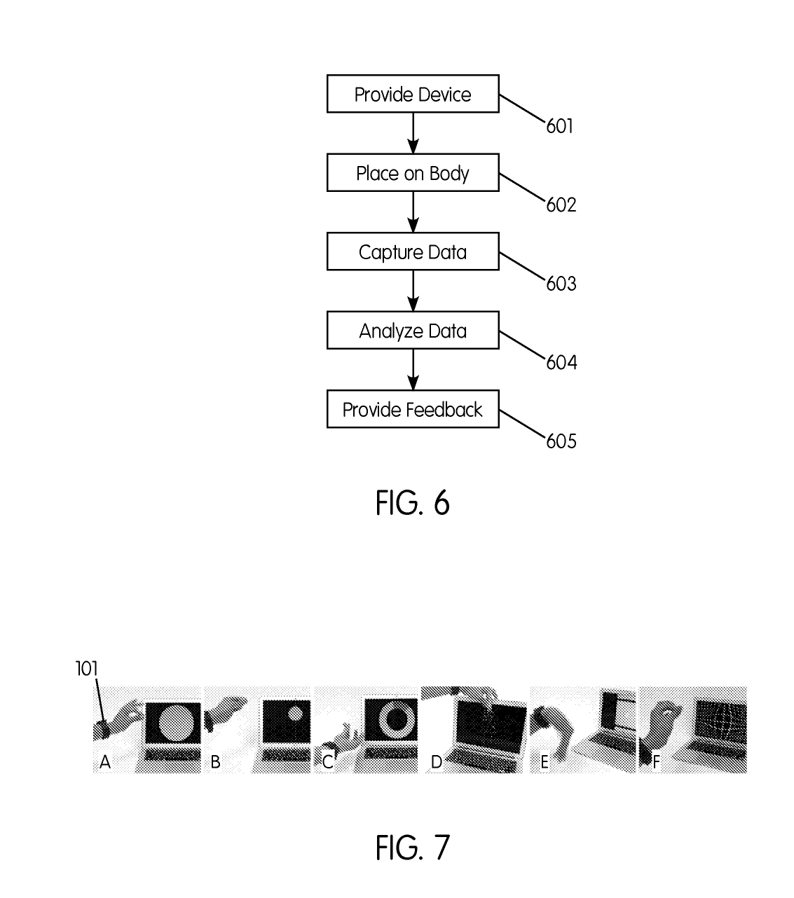

FIG. 6 is a flow diagram showing the method, according to one embodiment. In step 601, a wearable electronic device 101 capable of capturing data at a rate of about 4000 Hz is provided. At step 602, the wearable 101 is placed on a first body part. Next, during step 603, data is captured by the vibration sensor 102. The data is related to movement of a body part at a distance from the body part in contact with the wearable 101. For the example using a smartwatch, the wrist would be the first body part and the hand or fingers would be the moving body part. At step 604, the data is analyzed. This step could simply be determining whether the data is structured vibrational data or a hand movement. Finally, at step 605, the user is provided feedback through the wearable 101. The feedback can include the action of launching an application, providing an audible cue, or simply displaying a message on the screen.

Once the bio-acoustic signals are received on the wearable 101, several signal processing operations can be completed to detect and classify hand gestures in real-time. For each incoming signal frame t, the power spectra of the fast Fourier transform (FFT) is computed on data from each accelerometer axis, producing three spectra Xt, Yt, Zt. Optionally, a Hamming window on the FFT is used to minimize spectral banding. To make sensing robust across hand orientations, the DC component is removed and the three FFTs combined into one by taking the max value across the axes (F_{t,i}=max(X_{t,i}, Y_{t,i}, Z_{t,i})).

Next, the average of the w=20 past FFT spectra (Sᵢ=F_{t-1,i}, . . . , Ft_{t-w+1,i})) is computed and statistical features are extracted from the averaged signal: mean, sum, min, max, 1st derivative, median, standard deviation, range, spectral band ratios, and the n highest peaks (n=5). These features form the input to a SMO-based support vector machine (SVM) (poly kernel, ε=10⁻¹², normalized) for real-time classification. In this example embodiment, the band ratios, peaks, mean, and standard deviation are capable of providing 90% of the bio-acoustic signal's discriminative power. Table 1 describes these features and the motivations behind their use.

TABLE 1

| Feature Set | Operation | Justification |
|---|---|---|
| Power spectrum | Sᵢ | Specific frequency data |
| Statistical | μₛ, σₛ, Σₛ, max(S), | Characterizes gross features of FFT |
| | min(S), centroid, | signal |
| | peaks | |
| 1^{st} Derivative | $\frac{d}{dt} ( {S}_{t + 1} ) = {S}_{t + 1} - {S}_{t}$ | Encodes signal peaks and troughs |
| Band Ratios | ${B}_{j , k} = \frac{{S}_{j}}{{S}_{k}}$ | Describes overall FFT shape, power distribution |

When hand gestures are combined with relative motion tracking (e.g., native data from IMUs 102), the example embodiment uncovers a range of interaction modalities (see FIG. 7). These include: buttons, sliders, radial knobs, counters, hierarchical navigation, and positional tracking.

In another example embodiment, the method of the present invention can be used to identify grasped objects 301. With objects identified, context-relevant functionality or applications can be launched automatically by the wearable electronic device 101. For example, when a user operates a mechanical or motor-powered device, the object 301 produces characteristic vibrations, which transfer into the operator. The wearable electronic device 101 is able to capture these signals, which can be classified, allowing interactive applications to better understand their user's context and further augment a wide range of everyday activities.

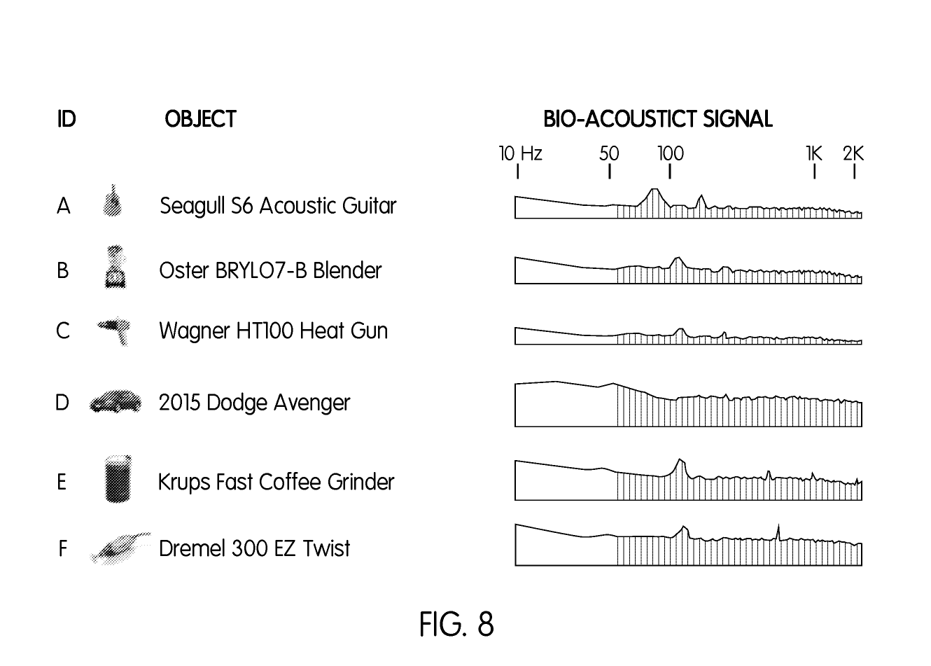

The same signal processing pipeline used for gestures is used for object detection, but with slightly tweaked parameters (w=15, n=15). In addition, the data analysis step comprises a simple voting mechanism (size=10) to stabilize the recognition. The method recognizes a wide range of objects 301 (see FIG. 8), expanding capabilities for rich, context-sensitive applications.

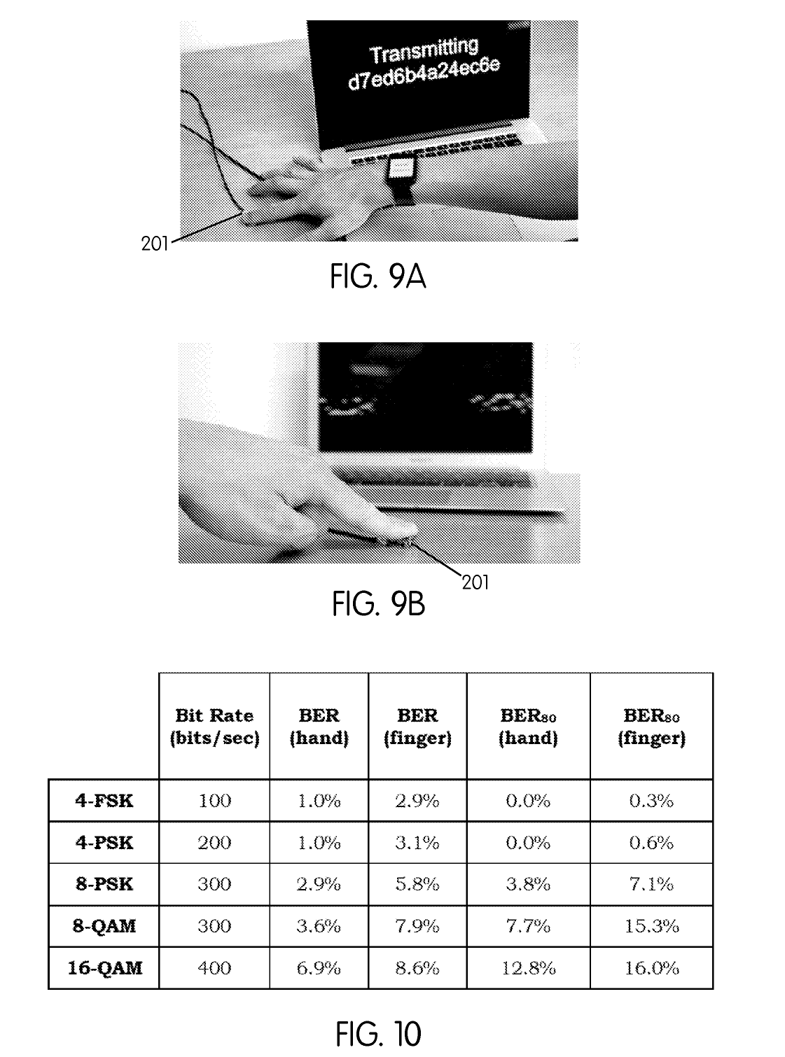

In yet another alternative embodiment, the method of the present invention can be used to augment environments and objects with structured vibrations. For example, in one embodiment a “vibro-tag” 201 comprising a small (2.4 cm³) SparkFun COM-10917 Bone Conductor Transducer, powered by a standard audio amplifier, is used to augment a user's environment. When a user touches the tag 201, modulated vibrations are transmitted bio-acoustically to the wearable electronic device 101, which decodes the acoustic packet and extracts a data payload (see FIGS. 9A-9B). Such tags 201 can be used much like RFID or QR Codes while employing a totally orthogonal signaling means (vibro-acoustic). A unique benefit of this approach is that it is only triggered upon physical touch (i.e., not just proximity) and is immune to variations in lighting conditions, for example.

In one embodiment, the vibro-tags 201 are inaudible to the user, but still capable of transmitting data at high speed. Because the IMU 102 can only sense frequencies up to 2 KHz, ultrasound frequencies (e.g. frequencies above 16 kHz) cannot be used. Further, frequencies above 300 Hz are not used as they would manifest as audible “buzzing” sounds to the user. As a result, in one embodiment, 200 Hz is utilized as a suitable carrier frequency for data transmission. However, a person having ordinary skill in the art will appreciate that other frequencies can be used, particularly if audible sounds are tolerable.

In one example embodiment, the data transmission system is a full stack signal pipeline, consisting of data packetization, error detection, error correction, and modulation layers. The input data stream is segmented into individually transmitted data packets. In one example, the format comprises an 8-bit sequence number combined with a data payload. Packet size is constrained by the error detection and correction layers; in this embodiment, it can be up to 147 bits in length. In order to detect transmission errors and ensure that bad data is not accidentally accepted, an 8-bit cyclic redundancy check (CRC) is optionally appended to the message. In this example, the CRC is computed by truncating the Adler-32 CRC of the message.

Next, error correction is applied. Although this stage also detects errors (like the CRC), its primary purpose is to mitigate the effects of minor transmission problems. In an example embodiment, a Reed-Solomon code is used with 5 bits per symbol, allowing the system to have 31 symbols per message (a total of 155 bits). These parameters were chosen to allow a single message to be transmitted in approximately one second using common modulation parameters. The number of ECC symbols can be tuned to compensate for noisier transmission schemes.

At this point, the full message+CRC+ECC is transmitted, totaling 155 bits, as modulated vibrations. Four different modulation schemes can be used, using binary Gray coding to encode bit strings as symbols:

Amplitude Shift Keying (ASK): data is encoded by varying the amplitude of the carrier signal;

Frequency Shift Keying (FSK): data is encoded by transmitting frequency multiples of the carrier signal;

Phase Shift Keying (PSK): adjusting the phase of the carrier signal, with respect to a fixed reference phase; and

Quadrature Amplitude Modulation (QAM): data encoded as variations in phase and amplitude, with symbols encoded according to a constellation diagram mapping phase and amplitude combinations to bit sequences.

In an alternative embodiment, the message is created with a short header sequence consisting of three 20 ms chirps at 100 Hz, 300 Hz, and 200 Hz. This sequence is readily recognized and quite unlikely to occur by accident. Furthermore, the presence of a 300 Hz chirp in the header prevents accidental detection in the middle of a transmission. Finally, the 200 Hz chirp provides a phase and amplitude reference for the ASK, PSK and QAM transmission schemes, eliminating the need for clock synchronization between the tag 201 and wearable 101.

Decoding can be performed on the wearable electronic device 101 itself, using an optimized decoding routine. The decoder 106 continuously reads samples from the accelerometer or IMU 102, converts the samples to 6400 Hz (to simplify FFT computations), and continuously searches for the header sequence. When found, the decoder 106 demodulates the signal (using the amplitude and phase of the 200 Hz header chirp), performs decoding, verifies the CRC, and reports the resulting message to an application (if decoding was successful).

In an example demonstration of the method of the present invention, 18 participants (10 female, mean age 25.3, 17 right-handed) were recruited for a live user study. Participants were asked to perform a series of tasks while wearing a wearable electronic device 101. Since variations in user anatomy could affect bio-acoustic signal propagation, the user's body mass index (BMI, mean=22.3) was recorded to further explore the accuracy of the sensing technique. To verify the robustness of the method across different devices 101, the study used two different devices 101 of the same model (Watch A and Watch B), randomized per user. All machine learning models were trained on Watch A, but deployed and tested on both watches 101.

To test the accuracy of gesture recognition, different machine learning models were trained for each gesture set (FIG. 5). Each model was calibrated per-participant, i.e., models were trained for each user. Across all 17 users and 17 gestures (in all three gesture sets), the method achieved a mean accuracy of 94.3% (SD=4.1%). There were no statistically significant differences between users and their BMI.

For opbject detection, data was collected from one user on 29 objects using a single wearable electronic device 101. The collected data was then used to train a machine learning model. An example object set and their bio-acoustic signatures are shown in FIG. 8.

After collecting the data from a single user, real-time object classification was performed for all 17 participants using the same 29 objects 301. Objects were spread across six locations to vary environmental conditions. These locations include: personal desk area, shared woodshop, office, kitchen and bathroom, public common area, and a parking space. Further, all objects 301 were tested in a location that was different from where it was trained. A single trial involved a user interacting with one of the 29 objects 301. Participants were briefly shown how to operate the objects 301 (for safety), but were free to grasp the object however they wished. Objects 301 were randomized per location (rather than randomized globally).

Across 29 objects 301, 17 users, and using data that was trained on a single person four weeks prior, an overall object detection accuracy of 91.5% (SD=4.3%) was obtained. Two outlier objects 301 were found that were 3.5 standard deviations below the mean. When these two outlier objects 301 are removed, the method returned an overall accuracy of 94.0% (27 objects), with many objects 301 achieving 100% accuracy. Additionally, no statistical differences were found between a user's body-mass index or object 301 location. Overall, these results suggest that object detection is indeed accurate and robust across users and environment, and object bio-acoustic signatures are consistent over time.

In another example embodiment, the method recognizes structured vibrations that can be used with several variations of ASK, PSK, FSK and QAM modulation schemes. In addition, multiple symbol rate and bits-per-symbol configurations can be used. For example, configuration can include: 4-FSK (2 bits per symbol, transmitting frequencies of 50, 100, 150 and 200 Hz), 4-PSK (2 bits per symbol), 8-PSK (3 bits per symbol), 8-QAM (3 bits per symbol, non-rectangular constellation), 16-QAM (4 bits per symbol, non-rectangular constellation).

Using these various schemes, 1700 trials were collected with a bit error rate results, which compares the received, demodulated message with the original transmitted message. (See FIG. 10). Raw bit transmission rate indicates the modulation method's data transmission speed, while bit error rate (BER) indicates the percentage of bits in the received message that were incorrect. The bit error distribution has a significant long tail across all conditions: most messages are received correctly, but a small number of messages are received with many errors.

The 80^{th} percentile BER (BER₈₀), for parity with Ripple, is used to get a better sense of the distribution. This measurement has a practical impact on the choice of error correction parameter: if an error correction scheme is chosen that can correct errors up to BER₈₀, then it can be expected to successfully decode 80% of transmitted packets.

The results indicate that 4-PSK provides optimal performance in terms of BER across all conditions, when considering the raw bit rate. With a BER₈₀ of 0.6% (0.93 message bits), only 2 Reed-Solomon ECC symbols would need to be added to our message in order to correct 80% of messages, leaving 137 bits for the payload. This payload takes 0.83 seconds to transmit (155 bits at 200 bits per second, plus header overhead), for an overall transmission rate of 165 bits per second (with a 20% packet loss rate), through the finger, hand and wrist.

In a system that takes advantage of accelerometers and IMUs 102, it is critically important to reduce the detection of false positives (i.e., an action that is unintentionally triggered). To validate the resistance of the method to false positives, the classifier is trained with a large set of background data (i.e., negative training examples). In this example, 17 participants were asked to perform several mundane and physically rigorous activities in different locations. These activities included: walking for two minutes, jogging in place for 30 seconds, performing jumping jacks for 30 seconds, reading a magazine or book for one minute, and washing hands for 30 seconds. These five activities were randomly interspersed throughout the object detection study (i.e., when users transitioned between each of the six building locations).

While participants performed these activities, the number of “false detections” triggered by the system (any prediction that was not “null” or “no object” was considered a false positive) were tallied. Across 17 users, six random locations, and five activities, collectively spanning a total of 77 minutes, the method triggered a total of six false positive classifications. For 12 of 17 participants, the system triggered no false positives. These results suggest that false positives can be greatly reduced by exposing the machine-learning model to a large set of negative examples.

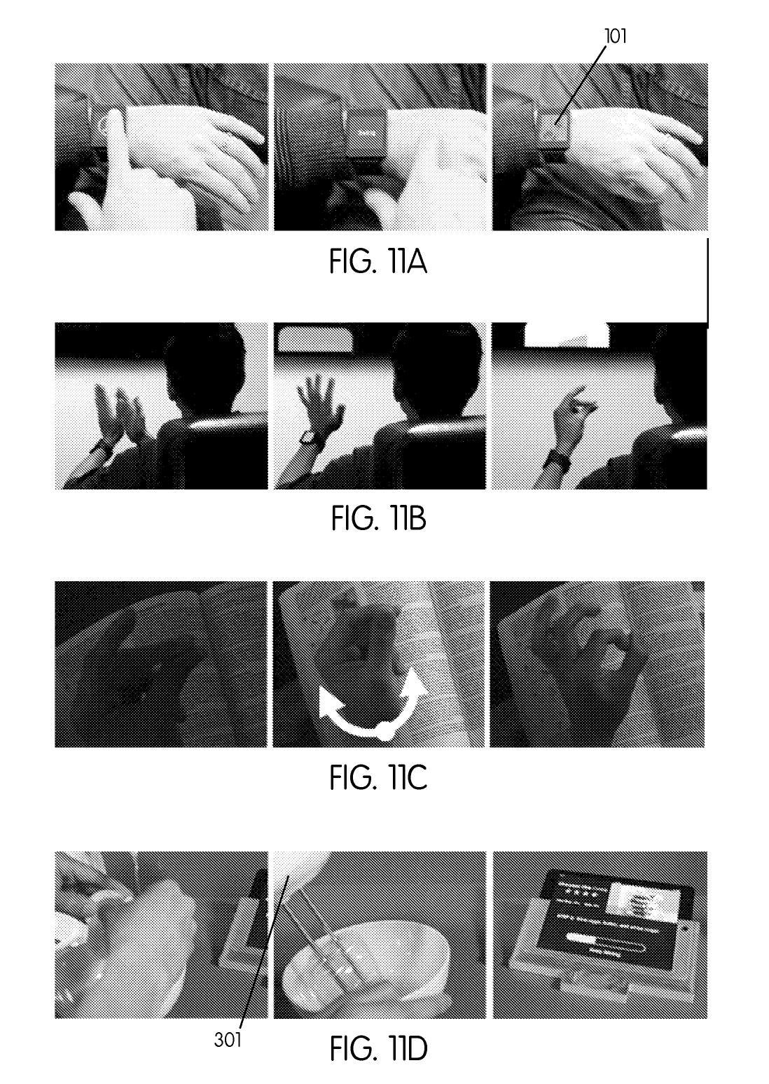

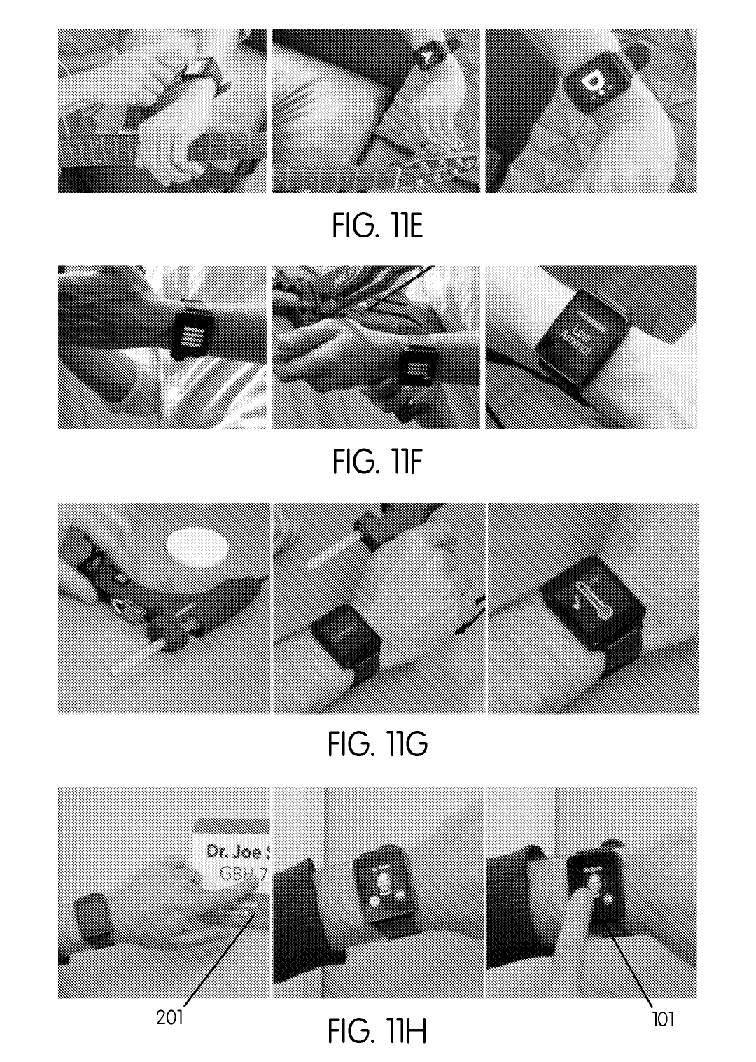

The methods described herein open the possibility for enhanced interaction with wearable electronic devices 101. Hand gestures can be used to appropriate the area around the watch for input and sensing. For example, in a smartwatch launcher, navigation controls can be placed on the skin (e.g., left, right, select), as well as enabling users to traverse back up through the hierarchy with a flick gesture (FIG. 11A).

Other examples of interaction can include the following. Gestures can be used to control remote devices. For example, a user can clap to turn on a proximate appliance, such as a TV; wave gestures navigate and snaps offer input confirmation. Flick gestures can be used to navigate up the menu hierarchy (FIG. 11B).

Gestures can also be used to control nearby infrastructure. For example, a user can snap his fingers to turn on the nearest light. A pinching gesture can be used as a clutch for continuous brightness adjustment, and a flick confirms the manipulation (FIG. 11C).

Because the method of the present invention can also be used to identify objects 301, applications offer the ability to better understand context and augment everyday activities. For example, the kitchen experience can be augmented by sensing equipment used in the preparation of a meal and e.g., offering a progress indicator for blending ingredients with an egg mixer (FIG. 11D). Of note, the feedback provided once the object is recognized is on a device separate from the wearable 101.

The method can also sense unpowered objects 301, such as an acoustic guitar. For example, the method can detect the closest note whenever the guitar is grasped, and provide visual feedback to tune the instrument precisely (FIG. 11E). Detection happens on touch, which makes it robust to external noise in the environment.

Through object sensing, the method can also augment analog experiences with digital interactivity. For example, with a Nerf gun, it can detect the loading of a new ammo clip, and then keep count of the number of darts remaining (FIG. 11F).

Many classes of objects 301 do not emit characteristic vibrations. However, with a vibro-tag 201, the object can emit inaudible, structured vibrations containing data. For example, a glue gun (non-mechanical but electrically powered) can be instrumented with a vibro-tag 201. The tag 201 broadcasts an object ID that enables the wearable 101 to know what object 301 is being held. It also transmits metadata e.g., its current temperature and ideal operating range (FIG. 11G).

Structured vibrations are also valuable for augmenting fixed infrastructure with dynamic data or interactivity. For example, in an office setting, a user can retrieve more information about an occupant by touching the room nameplate augmented with a vibro-tag 201, which transmits e.g., the person's contact details to the wearable 101 (FIG. 11H).

While the disclosure has been described in detail and with reference to specific embodiments thereof, it will be apparent to one skilled in the art that various changes and modification can be made therein without departing from the spirit and scope of the embodiments. Thus, it is intended that the present disclosure cover the modifications and variations of this disclosure provided they come within the scope of the appended claims and their equivalents.

What is claimed is:

1. A method of interacting with a wearable electronic device comprising:
providing a wearable electronic device, the wearable electronic device comprising an inertial measurement unit capable of capturing data at a rate of about 4000 Hz or more;
placing the wearable electronic device in contact with a first body part;
capturing data related to a movement of a second body part, wherein the movement creates vibrations that travel from the second body part to the inertial measurement unit of the wearable electronic device;
analyzing the data; and
providing feedback though the wearable electronic device based on the analyzed data.

2. The method of claim 1, further comprising:
classifying the movement based on the analyzed data.

3. The method of claim 1, wherein the movement comprises a hand gesture.

4. The method of claim 1, wherein the movement comprises motion created by an object touching the second body part.

5. The method of claim 1, wherein the vibrations have a frequency greater than 200 Hz.

6. The method of claim 1, wherein the IMU comprises at least one of an accelerometer and a gyroscope.

7. The method of claim 1, wherein the wearable electronic device is a smart-watch.

8. The method of claim 4, wherein the object is a transducer emitting a structured vibration.

9. The method of claim 8, where the structured vibration comprises a header sequence followed by a message.

10. The method of claim 9, wherein the header sequence comprises chirps at 100 Hz, 200 Hz, and 300 Hz.

11. The method of claim 1, wherein analyzing the data comprises:
extracting a maximum value at a plurality of frequency bands.

12. The method of claim 8, wherein the structured vibration comprises a data packetization layer, an error detection layer, an error correction layer, and a modulation layer.

13. The method of claim 1, wherein analyzing the data comprises:
determining a power spectra of a fast Fourier transform for each axis of a three-axis accelerometer in the inertial measurement unit;
combining the power spectra of each axis into a combined power spectra by using a maximum value of the three axis.

14. A system for providing interaction between a user and a wearable electronic device comprising:
a wearable electronic device comprising an inertial measurement unit capable of operating at about 4000 Hz,
wherein the inertial measurement unit outputs data related to bio-acoustic vibrations received at the wearable electronic device;
a classifier for correlating the data with at least one of a hand gesture, grasped object, or structure vibration.

15. The system of claim 14, further comprising:
a vibro tag that outputs the structured vibration.

16. The system of claim 15, wherein the vibro tag comprises a transducer operating at about 100-300 Hz.
